(19) United States
(12) Patent Application Publication
(10) Pub. No.: US 2010/0189755 A1
(43) Pub. Date: Jul. 29, 2010

(54) Dryer Sheets and a Method for Treating Skin

(75) Inventors: Markus Doerr, Glenmoore, PA (US); J. Michael Ogden, Saint Leon sur L'isle (FR)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Duesseldorf (DE)

(21) Appl. No.: 12/632,285

(22) Filed: Dec. 7, 2009

Related U.S. Application Data
(62) Division of application No. 11/204,882, filed on Aug. 16, 2005, now abandoned.
(60) Provisional application No. 60/602,468, filed on Aug. 18, 2004.

(51) Int. Cl. A61K 9/70; A61K 31/225; A61K 31/23; A61K 31/14; A61K 33/06; A61K 31/355; A61K 36/886; A61K 31/715; A61P 17/00

(52) U.S. Cl. 424/402; 514/547; 514/552; 514/642; 424/682; 514/458; 424/744; 514/58

(57) ABSTRACT

The invention relates to substrates impregnated with the skin-treating components which when introduced into a tumbling-type dryer in which it contacts fabric articles, the skin-treating component is transferred to the fabric articles which, when contacted with the skin of a user applies the skin-treating component to human skin.

### CROSS-REFERENCE TO RELATED APPLICATIONS

This application claims priority from Provisional Application Ser. No. 60/602,468 filed Aug. 18, 2004, the entire contents of which is incorporated herein by reference.

### STATEMENT REGARDING FEDERALLY SPONSORED RESEARCH OR DEVELOPMENT

Not applicable.

### BACKGROUND OF THE INVENTION

The present invention relates to a system and method for treating skin, and, more particularly, to a skin-treating system and a substrate which is effective to absorb a composition, retain it in liquid or dry form therein and release an effective amount of the composition onto or into clothing or other articles which in use contact the skin, within a clothes dryer in the course of a drying cycle.

The treatment of fabric with certain types of chemical compounds to impart softness when washing articles of clothing has been commonly employed in households, commercial laundromats and in the textile industry. The term “softness” refers to a quality of the fabric in which its “hand” or feel to the touch is smooth, pliable and fluffy, as distinguished from coarse or scratchy. In addition to softening agents, chemical compounds used in washing clothing often include anti-static agents to reduce the static cling of the fabric. The term “static cling” refers to the tendency of articles of clothing to adhere to a wearer after being dried in a clothes dryer as a result of static electrical charges created on the surface of the fabric. The static charges cause the fabric to repel itself; however, such electrostatic charges can attract lint and dust to the fabric. The treatment of articles which contact the skin with softening agents and anti-static agents increases their comfort when worn, and generally reduces wrinkles which makes ironing easier.

There are many patents and articles directed to dryer sheets which improve the feel, softness and electrostatic properties of articles during the drying cycle. Representative disclosures appear in U.S. Pat. No. 5,658,651 and WO 03/087465, the entire contents of each reference are incorporated herein by reference. However, these dryer sheets do not carry components which are intended to provide benefits to the skin. U.S. Pat. No. 6,623,746 (the entire contents of which is incorporated herein by reference) discloses PIT emulsions which are applied to tissues made from recycled paper, to soften the paper for contact with body parts. The softening agents applied to the paper generally comprise emollients and emulsifying agents to permit easy application of the softening composition to the tissue. The tissues are relatively thin substrate like facial tissues, toilet tissue, paper handkerchiefs, make-up removing wipes, freshening wipes, kitchen rolls, and the like. The softened paper articles are not intended for dryer use, being of generally light weight and low strength.

### SUMMARY OF THE INVENTION

While the use of fabric treating compositions in dryer sheets is known in the industry, the present invention is directed to the use of skin-treating compositions which are transferred onto an article, which will contact the skin of a user, from a dryer sheet during a drying cycle, and then subsequently applied onto human skin when the article is worn by an individual. The skin-treating compositions are intended to contain ingredients which impart beneficial properties to human skin.

The present invention is directed to a dryer sheet comprising a substrate impregnated with a skin-treating composition containing:
(a) a skin-treating component;
(b) an emulsifier;
(c) optionally, a carrier; and
(d) optionally, auxiliaries and additives.

The present invention is also directed to a process for treating human skin comprising:
(a) providing a dryer sheet comprising a substrate impregnated with a skin-treating composition containing:
   (i) a skin-treating component;
   (ii) an emulsifier;
   (iii) optionally, a carrier; and
   (iv)optionally, auxiliaries and additives;
(b) providing a clothes dryer which provides tumbling action;
(c) providing articles which contact the skin of a user during use;
(d) inserting the dryer sheet and the article, which contacts the skin of a user during use, into the clothes dryer;
(e) actuating the clothes dryer in order to produce tumbling within the dryer to transfer at least a portion of the skin-treating component to the article to form a skin-treating article;
(f) removing the skin-treating article from the clothes dryer; and
(g) contacting human skin with the skin-treating article.

### DETAILED DESCRIPTION OF THE INVENTION

All numbers expressing quantities of ingredients and/or reaction conditions are to be understood as being modified in all instances by the term “about”.

The skin-treating system of this invention comprises the combination of a skin-treating composition and a substrate capable of releasably absorbing or adsorbing the composition in the form of a dryer sheet. The substrate, which retains the skin-treating composition therein, is introduced into a clothes dryer along with the article to be treated wherein the composition is released onto the fabric of the article. Each component of the system of this invention is described separately below, followed by illustrative examples.

The skin-treating composition of the present invention is prepared by mixing, in desired proportions, the following: (a) a skin-treating component; (b) an emulsifier; (c) optionally, a carrier; and (d) optionally, auxiliaries and additives.

The skin-treating component can comprise any known ingredient for treating human skin. Examples thereof include, but are not limited to, insect repellants, UV absorbers, skin moisturizers, tanning agents, wrinkle removers, deodorants, cellulite reducers, vitamins, anti-oxidants, minerals, lipid layer enhancers, hair growth suppressants, emollients, botanical actives, and the like.

The skin-treating component can comprise mixtures of skin-treating components which have the same or different functions. Depending on the nature of the skin-treating components, one skin-treating component can be a carrier for one or more other skin-treating components.

The useful skin-treating components must be in a form in which they can be releasably absorbed or adsorbed on a sheet-like substrate and readily transferred to the article to be treated in the dryer. A portion of the skin-treating component should be readily transferable to the article by the tumbling action of the dryer and direct contact of the impregnated substrate with the wet article. Preferably the skin-treating component is transferable from the sheet-like substrate to the article at ambient temperatures or temperatures in a range of 30° C. to 90° C., and preferably in a range of 30° C. to 75° C., and most preferably at a temperature in a range of 35° C. to 45° C. Although the temperature of the drying gas to the dryer can be in the range of 100° C. or higher, the temperature of the article being dried, and the sheet-like substrate can be considerably lower due to the evaporation of water and intimate contact between the articles being dried and the sheet-like substrate.

The sheet-like substrate and the skin-treating component can be formulated so that the skin-treating component can be transferred to the article at ambient temperature. Low temperature transfer (30° C.-50° C.) is desirable since certain fabrics can be damaged at elevated temperatures over extended time periods and for the most part, the articles are in the low temperature range for most of the drying cycle.

The sheet-like substrate must be sufficiently strong to maintain its integrity during the tumbling time with the articles in the dryer especially when the articles are wet. In addition, the sheet-like substrate must be sufficiently absorbent or adsorbent and of a sufficient thickness and planar dimensions to carry a sufficient amount of the skin-treating composition to treat the weight of articles to be contacted with the impregnated substrate within the dryer. The substrate has a weight per square meter of from about 10 grams to about 500 grams and preferably from 25 grams to 250 grams per square meter.

The substrate is impregnated with the skin treating compositions in a ratio of 1:1 to 1:15 (weight ratio of substrate to skin treating composition), preferably 1:2 to 1:12 and most preferably 1:3 to 1:8.

The substrate is preferably formed from fibers, which can be woven or non-woven. The fibers can be natural fibers or synthetic fibers and the fibers can be treated to be compatible with the composition with which it is impregnated. That is, if synthetic fibers are utilized, the fibers can be hydrophobized or hydrophilized to more readily accept hydrophobic or hydrophilic compositions. The substrate can also be in the form of foamed materials or perforated materials which can contain cavities for carrying the skin-treating composition.

The thickness of the substrate is determined by the density of the substrate, the weight of the skin-treating component to be impregnated into the substrate and the strength required to withstand the tumbling action and contact with the articles being treated. Preferably, the substrate is formed from biodegradable materials which have a substantial capacity for impregnation with the skin-treating composition.

Since the skin-treating composition is transferred to the articles mainly by direct contact between the article and the impregnated substrate, the substrate must be of a size which can be readily tumbled with the articles being dried and circulate among the articles to provide sufficient contact between the skin-treating component carried by the substrate and the articles so that the skin-treating component can be transferred from the substrate to the articles. Substrates in the range of from about 12 to about 26 centimeters wide and from about 12 to about 34 centimeters long can be useful. The substrate may be from 0.3 mm to 3.0 mm thick and preferably 1.0 mm to 1.0 mm thick. For the smaller size substrates, it may be necessary to introduce more than one impregnated substrate into the dryer which enhances the achievement of contact between the impregnated substrate and articles. Generally, from about 0.5 grams to about 20 grams of skin-treating composition impregnated on the substrate is required per kilogram of articles based on the dry weight of the articles; preferably from about 1 gram to about 10 grams of skin-treating composition per kilogram of dry articles.

The substrate can be formed from a woven or non-woven fiber matrix. Preferably due to costs and availability, non-woven substrates of readily biodegradable material are preferred. A particularly useful fiber is a material sold under the trademark TENCEL®, Lenzing Fibers, Inc. of Lenzing, Austria. TENCEL® fibers are particularly useful since they have a tensile strength when wet of about 85% of the dry tensile strength and can absorb liquid up to about 800% of their dry weight and provide a highly liquid absorbent substrate in the form of a non-woven sheet which can be hydro-entangled or needle-entangled.

The substrate useful in the practice of the present invention preferably comprises a fiber matrix to which added strength is provided by weaving, or in the case of non-woven fiber substrates strength can be provided by hydro- or needle-entanglement or bonding where the random fibers are in contact. The structure of the substrate should be arranged so that it readily absorbs or adsorbs the skin-treating composition and readily releases the skin-treating composition to the tumbling articles in a dryer. Generally paper is not a suitable substrate due to its low wet strength and absorbent properties without special treatment to improve its strength and loft.

The use of a non-woven sheet formed from highly absorbent or adsorbent fibers provides superior performance in releasing the skin-treating composition onto the articles within a clothes dryer. Highly absorbent fibers are effective to trap or retain the composition in liquid form within the structure of the fibers, and then gradually release the composition during the course of a drying cycle under the influence of the tumbling action of the dryer and the application of heat. As a result, the composition is relatively uniformly released onto the fabric of the article over a comparatively long period of time, to distribute the composition more evenly and completely to the articles within the dryer.

The articles to be treated in the present invention comprise fabric articles which during use can contact the skin of a user; articles such as towels, sheets, pillowcases, underclothing, pajamas, shirts, blouses, pants, skirts, socks, stockings, handkerchiefs, dressing gowns, bathrobes, and the like. Generally any fabric article which contacts the skin can be treated with the skin-treating composition and process of the invention.

The skin-treating components can be formulated for impregnation into the substrate in the form of solutions, emulsions (w/o and o/w), and dispersions. The skin-treating composition can be in the form of a liquid or a solid at ambient temperatures as long as it can be transferred from the substrate to the articles during the drying cycle. That is, the skin-treating component can be a low-melting point material which becomes a liquid at the drying temperature and readily transfers from the substrate to the articles being dried. A preferred embodiment of the invention comprises a substrate impregnated with a solution, emulsion or dispersion of the skin-treating component absorbed or adsorbed onto a substrate formed from fibers, preferably a non-woven substrate.

It is not certain how the skin-treating component is transferred from the impregnated substrate to the article being dried, but it is believed that the transfer occurs diffusion controlled by direct contact between the impregnated substrate and the wet material being dried. An emulsifier in the composition aids in transfer of the skin-treating composition from the impregnated substrate to the damp article during the drying process. In addition, the emulsifying agent is believed to add softness to the articles without decreasing the water-absorbency as occurs with softeners used in well-known fabric softener dryer sheets.

As briefly discussed above, the skin-treating component can comprise a single material or a mixture of materials which are known to provide benefits to the skin. The skin-treating components can be derived from a broad range of classes of materials such as emollients, lubricants, super-fatting agents, natural extracts which provide benefits to the skin depending upon the nature of the natural extracts, the lees from wine-making, DNA derivatives, hydrolyzed proteins both of animal and vegetable origin, derivatives of the hydrolyzed proteins, plant extracts, the skin-active portions of plant extracts, and the like. The above classes of materials are general and non-limiting since the skin-treating components only need be in a form in which they can be impregnated onto the substrate and are releasably transferable from the impregnated substrate to the fabric article in contact with the impregnated substrate during the dryer operation. The skin-treating component can be impregnated onto the substrate in the form of a PIT emulsion of the oil/w or w/oil type. The PIT emulsions are generally of a fine particle size and, in addition, have a relatively low viscosity.

The skin-treating component can be a fatty acid alkyl ester of the formula R¹CO—OR² in which R¹CO is a linear or branched, saturated or unsaturated acyl group containing 8 to 22, preferably 12 to 18, carbon atoms and R² is a linear or branched alkyl and/or alkenyl group containing from 1 to about 22 carbon atoms. Preferably R² is a alkyl or alkenyl group containing from 6 to about 18 carbon atoms. Typical examples are esters of acrylic acid, isononanoic acid, capric acid, lauric acid, myristic acid, palmitic acid, palmitolaic acid, stearic acid, isostearic acid, oleic acid, elaidic acid, petroselic acid, elaeostearic acid, arabic acid, gadoleic acid, behenic acid, and urucic acid. Other esters can be esters of acids such as linoleic acid, linolenic acid and the equivalent mono- and di-unsaturated acids with the range of 8 to about 22 carbon atoms. The carboxylic acids can be esterified with alcohols such as ethyl alcohol, propyl alcohol, ethyl hexyl alcohol, capric alcohol, lauryl alcohol, stearyl alcohol, isostearyl alcohol, oleyl alcohol, elaidyl alcohol, petroselinyl alcohol, linolyl alcohol, linolenyl alcohol, behenyl alcohol, and the like. Esters having a plastic consistency are particularly useful for the practice of the present invention. Esters such as myristyl myristate, cetylearyl isononanoate, cetyl palmitate, cetyl stearate and the like are useful.

Fatty alcohols of the formula R³OH (II) in which R³ is a linear or branched alkyl or alkenyl group containing from about 8 to 22, preferably 12 to 18, and more preferably 14 to 16 carbon atoms are useful. These alcohols are well known in the art and are generally prepared by hydrogenation of a carboxylic acid with an equivalent number of carbon atoms.

Another class of skin-treatment components which are useful in the practice of the present invention include the alcohol polyglycol ethers which are the products of the addition of ethylene and/or propylene oxide onto fatty alcohols or oxo-alcohols which preferably correspond to the formula R⁴O(CH₂CHR⁵O)ₙH in which R⁴ is a linear or branched alkyl and/or alkenyl group containing 8 to about 22, preferably about 12 to 18, and more preferably 14 to 16 carbon atoms, R⁵ is hydrogen or methyl and n is a number of from 1 to 50. Typical examples of the adducts of an average of 1 to 50, preferably 5 to 40, and more preferably 10 to 20 mols of ethylene oxide with caprylic alcohol, capric alcohol, lauric alcohol, myristyl alcohol, cetyl alcohol, palmitoleyl alcohol, stearyl alcohol, isostearyl alcohol, oleyl alcohol, and the like. Adducts of 10 to 20 mols of ethylene oxide with technical fatty alcohols containing 16 to 18 carbon atoms, for example cetearyl alcohol or tallow fatty alcohol are preferred. These compounds can also act as emulsifiers of the nonionic type.

Another useful component which can act as a skin-treatment component and an emulsitifer in the skin-treating composition of the present invention can comprise partial glyceride esters of the formula R⁷CH₂CH(OH)CH₂OCOR⁶ wherein R⁶CO is a linear or branched, saturated or unsaturated acyl group containing 8 to 22, preferably 12 to 18, and more preferably 14 to 16 carbon atoms. R⁷ can be H or R⁶OCO. The partial glyceride esters, i.e. comprise monoglycerides, diglycerides, and technical mixtures thereof and may contain small amounts of triglycerides, depending upon the method of production. The typical examples are mono- and/or di-glycerides based on caprylic acid, capric acid, lauric acid, palmitic acid, palmitoleic acid, stearic acid, isostearic acid, oleic acid, elaidic acid, petroselic acid, elaeostearic acid, arachic acid, gadolaic acid, behenic acid, and erucic acid and technical mixtures thereof. Partial esters with a monoglyceride content of 50 to 90% by weight and preferably 60 to 90% by weight are preferred.

The skin-treatment component can contain known skin-care oils such as guerbet alcohols based on fatty alcohols containing 6-18 and preferably 8-10 carbon atoms, esters of linear carboxylic acids with linear C₆₋₂₂ fatty alcohols, esters of linear C₆₋₂₂ fatty acid with branched alcohols, more particularly 2-ethylhexanol, esters of hydroxycarboxylic acids with linear or branched C₆₋₂₂ fatty alcohols and diesters such as dioctyl malate, esters of linear and/or branched fatty acids with polyhydric alcohols, for example propylene glycol dimer diol or triol and/or Guerbet alcohols, triglycerides based on C₆-₁₀ fatty acids, liquid mono, di-esters based on C₆₋₁₈ fatty acid, esters of C₆₋₂₂ fatty alcohol, and/or Guerbet alcohols with aromatic carboxylic acids, more particularly benzoic acid, esters of C₂₋₁₂ dicarboxylic acids with linear or branched alcohols containing 1-22 carbon atoms or polyols containing 2-10 carbon atoms and 2-6 hydroxyl groups, vegetable oils, branched primary alcohols substituted cyclohexanes, linear and branched C₆₋₂₂ fatty alcohol carbonates, Guerbet carbonates, esters of benzoic acid with linear and/or branched C₆₋₂₂ alcohols (for example Finnsolv®), linear or branched, symmetrical or nonsymmetrical dialkyl ethers containing 6-22 carbon atoms per alkyl group, ring-opening products of epoxidized fatty acid esters with polyols, silicone oils and/or aliphatic or napthinic hydrocarbons. The skin-care oils are preferably used in a quantity of 10-50% by weight, based on the active substance content of the skin-treating component.

The skin-care composition can contain emulsifiers and co-emulsifiers, preferably nonionic, anionic, cationic, or amphoteric emulsifiers which are well known in the art. The emulsifiers and co-emulsifier can include emulsifiers such as C10-18 fatty acid monoesters and diesters of reaction products of 1-30 mols of ethyleneoxide with glycerol; glycerol mono/diesters, sorbitan mono/diesters and sugar mono/diesters of saturated and unsaturated fatty acid containing 6-22 carbon atoms or hydroxy carboxylic acids containing 2-6 carbon atoms, for example citric acid, malic acid or tartaric acid and ethyleneoxide addition products thereof; alkyl mono- and oligo-glycosides containing 8-22 carbon atoms in the alkyl group and ethoxylated analogs thereof; addition products of 15-60 mols of ethylene oxide with castor oil and/or hydrogenated castor oil; polyol esters and in particular, polyglycerol esters such as, for example, polyglycerol polyricinoleate, polyglycerol poly-12-hydroxy stearate or polyglycerol dimerate. Mixtures of compositions from several of the classes can also be useful. Other materials can be useful as coemulsifiers such as addition products of 2-15 mols of ethylene oxide with castor oil and/or hydrogenated castor oil; partial esters based on a linear, branched or, unsaturated or saturated C₆₋₂₂ fatty acids, ricinoleic acid and 12-hydroxy stearic acid with glycerol, polyglycerol, pentaerythritol, dipentaerythritol, sugar alcohols (for example sorbitol) alkyl glucosides (for example methyl glucoside, butyl glucoside, lauryl glucoside and the like), and polyglucosides (such as cellulose); mono-, di- and tri-alkyl phosphates and mono-, di-, and/or tri-PEG alkyl phosphates and salts thereof; wooll wax alcohols; polysiloxane/polyalkylpolyether copolymers and corresponding derivatives; mixed esters of pentaerythritol, fatty acids, citric acid and fatty alcohol according to DE-PS 1165574 and/or mixed esters of fatty acids containing 6-22 carbon atoms, methyl glucose and polyols, preferably glycerol or polyglycerol; and polyalkyleneglycols. Preferably nonionic emulsifiers are used.

Other useful emulsifiers are zwitterionic surfactants. Zwitterionic surfactants are surface-active compounds which contain at least one quaternary ammonium group and at least one carboxylate and one sulfonate group in a molecule. Particularly suitable zwiterrionic surfactants are the so-called betaines, and the like. Ampholytic surfactants are suitable co-emulsifiers.

Esterquats, are well-known emulsifiers which can be useful in the practice of the present invention.

The skin-treatment components can contain active substances such as chitosans, which are well known skin-treatment components and are prepared by known methods. (Deoxy)rybonucleic acids can be included in the skin-treatment composition since they are known to have inflammation-inhibiting and antioxidative properties. The (deoxy)ribonucleic acids include DNA, and RNA.

In preferred embodiments, the skin-treatment component may also include the mild surfactants, skin-care oils, superfatting agents, stabilizers, consistency factors, thickeners, polymers, silicone compounds, biogenic agents, deodorizers, film-formers, preservatives, hydrotropes, solubilizers, anti-oxidants, insect repellants, self-tanning agents, U/V filters, dyes, and the like.

Super-fattying agents are known substances, for example, lecithin, lecithin derivatives, polyol fatty acid esters, mono-glycerides and fatty acid alkyl amides, and fatty acid alkanol amides.

The skin-treating composition may contain thickeners which include polysaccharides such as xanthan gum, guar gum, agar-agar, alginates and tyloses, carboxy methyl cellulose, hydroxy ethyl cellulose and relatively high molecular weight polyethylene glycol monoesters and di-esters of fatty acids, polyacrylates, polyacrylamides, polyvinyl alcohol and the like.

The skin-treatment composition may also contain silicone compounds for example, di-methyl siloxane, methyl phenyl polysiloxanes, cyclic silicones and amino; fatty acid; alcohol; polyether-, epoxy-, fluorine-, glycoside- and/or alkyl-modified silicone compounds. Preferably the silicone compounds are in a liquid form at room temperature.

The composition of the present invention may contain biogenic agents such as for example tocopherol, tocopherol acetate, tocopherol palmitate, ascorbic acid, carotene, deoxiribonucleic acid, retinol, bis-abolol, alantoin, phytantriol, panthenol, AHA acids, amino acids, ceramides, pseudoceramides, essential oils, plant extracts, protoleic enzymes and vitamin complexes.

The skin-treating composition of the invention may additionally contain antiperspirants such as aluminum chlorhydrates, aluminum/zirconium salts, esterase inhibitors and the like. The skin-treating composition of the invention may also additionally contain peptides, lipids, nucleotides, nucleosides sulfoximine compounds, homocystine sulfoximine, butionine sulfones, alpha-hydroxy fatty acids, citric acid, lactic acid, malic acid, and derivatives thereof.

The composition may additionally contain hydrotropes such as ethanol, isopropyl alcohol, or polyols to improve flow behavior. Typical examples include glycerol, alkylene glycols such as ethylene glycol, diethylene glycol, propylene glycol, butylene glycol, hexylene glycol and polyethylene glycols with an average molecular weight of 100-1,000 Daltons; technical oligoglycerol mixtures with a degree of self-condensation of 1.5-10 such as for example technical diglycerol mixtures with diglycerol content of 40-50% by weight, sugar alcohols containing from 5-12 carbon atoms for example sorbitol or manitol, and sugars containing 5-12 carbon atoms such as fructose, glucose, sucrose and amino sugars such as glucamine.

The composition of the invention usually contains preservatives to prevent or reduce biodegradation and spoilage of the composition. Suitable preservatives include compounds and compositions which are well known in the art such as formaldehyde, parabens, pentanediol or sorbic acid and other classes of compounds which are well known in the art.

The composition can also contain insect repellants and the like. As an optional ingredient, the skin-treating composition of the invention may also contain perfume oil to enhance the odor of the composition and the odor of the articles to which it is applied.

UVA protection factors including UVA filters and UV-B filters which are now well-known materials can also be included in the skin-treatment composition useful in the skin-treating system of the invention.

Anti-oxidant materials may also be included in the composition to provide secondary protection factors such as anti-oxidants and the like.

Since the skin-treatment components useful in the practice of the present invention are primarily transferred by contact between the impregnated substrate to the articles to a large extent, the vapor pressure of the skin-treatment components is not important as long as the components can be readily transferred from the impregnated substrate to the articles by contact within the dryer.

A mixture of the skin-treatment components with the emulsifier and, optionally, a carrier and preservative is first prepared. If the skin-treatment component is a solid material at room temperature, it is preferable to prepare the skin treatment composition at an elevated temperature. The skin-treatment component or mixture of components is preferably mixed with an emulsifier and an emulsion formed which emulsion can contain the optional preservatives, perfumes and the emulsion impregnated onto the substrate by known means such as spraying, dipping, spreading and other known methods for impregnating absorbent or adsorbent substrates with a composition.

If the skin-treating composition comprising a mixture of skin-treating components and emulsifier, water and optionally a preservative is in the form of an emulsion, the emulsion is first prepared and then impregnated onto the substrate.

When the skin-treating component(s) are soluble in a carrier, a solution of the skin-treating component and an emulsifier may be formed in the carrier and the solution is impregnated onto the substrate. Although not required to form a dispersion or emulsion which is impregnated onto the substrate, the emulsifier is preferably included in the composition since it aids in transferring the skin-treating composition from the substrate to the article during the drying cycle.

The impregnating composition may comprise a mixture of the skin-treating component and an emulsifier with optionally a preservative and additives and auxiliaries for direct impregnation into the substrate.

The additives and auxiliaries are known components in dryer sheets which can be incorporated into the composition of the present invention to provide known benefits to the treated article, such as anti-static properties, pleasing aroma and improved shelf life and the like. However, the additives and auxiliaries are not required to provide the skin-treating properties to the articles which contact the skin of a user.

The system of the invention provides a readily available method of treating the skin without hand application of emollients, UV screens, anti-inflammation components, anti-aging components, and the like which can be oily compositions which require skill in application.

The composition used to impregnate the substrate imparts a softness to the treated article without need for the usual fabric softeners used in conventional dryer sheets. In addition, the component used to impregnate the substrate of the present invention softens the article without reducing the hydrophillic properties of the article as occurs with many of the known fabric softeners used in conventional dryer sheets. This property is unexpected in view of the oily nature of many skin-treating components.

The skin-treating component impregnated on the substrate may contain from 5% by weight to 95% by weight skin-treating components; from 0.1% by weight to 40% by weight of at least one emulsifier; from 0% by weight to 35% by weight of auxiliaries and additives; from 0% by weight to 95% by weight of a carrier; preferably, from 10% by weight to 75% by weight of at least one skin-treating component; from 0.5% by weight to 30% by weight of at least one emulsifier; from 20% by weight to 80% by weight of a carrier and from 4.5% by weight to 25% by weight of auxiliaries and additives; and most preferably, from about 15% by weight to about 50% by weight of at least one skin-treating component; from about 5% by weight to about 20% by weight of at least one emulsifier; from about 35% by weight to about 75% by weight of a carrier; and from about 10% by weight to about 20% by weight of auxiliaries and additives.

The carrier is usually water but can be other liquid components which provides a vehicle for impregnating the substrate with the skin-treating components and the emulsifier. The carrier can be one of the components of the skin-treating components or the emulsifier or mixture of emulsifiers which can provide a mixture which is liquid at the impregnation temperature and has a viscosity at which the impregnation is enabled.

Preferred skin-treating components include ethyleneglycol distearate, glycerin monooleate and cetyl palmitate.

Preferred emulsifiers and coemulsifers include glyceryl stearate, coco glycoside and ceteareth-12.

The preferred carrier comprises water which can include additives such as glycerol, water-soluble diols and other water-soluble materials. However, the carrier can also be a non-aqueous liquid material in which the skin-treating component and emulsifier are soluble or dispensible. The non-aqueous carrier can provide a non-aqueous skin-treating composition which can have advantages such as a constant viscosity in use which is not affected by evaporation of water during the drying cycle.

In some of the embodiments of the invention, water need not be present in the skin-treating composition and the impregnated substrate is in a non-aqueous form. Even though water is not present in the impregnating composition, an emulsifier is generally present since the emusifier aids in transferring the skin-treating composition from the impregnated substrate to the damp articles.

The present invention will be better understood from the examples which follow, all of which are intended for illustrative purposes only and are not meant to unduly limit the scope of the invention in any way.

### EXAMPLE I

A dryer sheet capable of imparting antiperspirant/deodorizing properties to human skin can be prepared by impregnating a non-woven substrate with a skin-treating composition containing 54.9% by weight water, 24.0% by weight of aluminochlorohydrate, 0.1% by weight of chitosan, and 25% by weight of EMULGADE® CM* (a trademark of Cognis Corp.). The components are mixed to form an emulsion and the emulsion impregnated onto a substrate. The preparation of the emulsion and the impregnation can be carried out at room temperature to provide a wet substrate for inclusion in a tumbling dryer with articles to be dried.

### EXAMPLE II

A dryer sheet capable of imparting UV-protecting properties to human skin can be prepared by impregnating a non-woven substrate with a composition containing 53.0% by weight of water, 20.0% by weight of EMULGADE® CM, 7.5% of EUSOLEX® CR, 6.8% by weight of EUSOLEX® HMS, 5.0% by weight of EUSOLEX® OS, 4% by weight of CETIOL® B, 2.0% by weight of EUSOLEX® 9020, 0.8% by weight of EMULGIN® HRE 40, 0.7% by weight of NIPAGUARD BPX, and 02.2% by weight of COSMEDIA SP.

EMULGADE® CM is an emulsifier concentrate comprising cetearylisononoate, ceteareth-20, cetearyl alcohol, glycerol stearate, glycerine, cetyl palmitate, and ceteareth-12.

EUSOLEX® OCR is a UV B absorber:

EUSOLEX® HMS a UV B absorber:

EUSOLEX® -OS, UV B absorber:

CETIOL® B, (dibutyl adipate) an emollient:

EUSOLEX® 9020, (butyl methoxy-dibenzol-methane a UV A absorber:

EMULGIN® HRE 40, (PEG-40 hydrogenated castor oil) a solubilizer:

NIPAGUARD BPX, (a mixture containing phenoxyethanol, methylparaben, polyparaben, 2-Bromo-2-nitropropane-1,3 diol) a preservative:

COSMEDIA SP(sodium polyacrylate), an emulsifier, thickener.

### EXAMPLE III

A skin-treating composition was prepared containing:

| | % wt |
|---|---|
| Water | 49.4 |
| PLANTATEX HCC | 50.0 |
| KATHON CG-ICP | 0.05 |
| Fragrance* | 0.5 |

*Givaudin UM 046904/00 floral fruity fragrance

Charge a mixing vessel with water. Add PLANTATEX® HCC with moderate agitation. After the mixture is uniform, add KATHON CG-ICP and fragrance. Continue mild agitation to prevent finished batch from separating. Separation will occur within a few hours after agitation is stopped.

The mixture was applied to a non-woven substrate (SX285 from Greenbay Nonwovens, 100 grams per square meter) at a weight ratio of about 1:6.5 (substrate: skin-treating composition).

The substrate impregnated with the skin-treating composition was placed in a dryer with wet articles to be dried at a ratio by weight of the impregnated substrate: the dry weight of the articles of 1:150, and the articles dried by hot air while tumbling in contact with the impregnated substrate.

### EXAMPLE IV

A skin-treating composition was prepared containing:

| | % wt |
|---|---|
| Water | 45.45 |
| PLANTATEX HCC | 50.0 |
| DEHYQUART A-CA | 4.0 |
| KATHON CG-ICP | 0.05 |
| Fragrance* | 0.5 |

*Givaudin UM 046904/00 floral fruity fragrance

Charge a mixing vessel with water. Add PLANTATEX® HCC and then DEHYQUART A-CA with moderate agitation. After the mixture is uniform, add KATHON CG-ICP and fragrance. Continue mild agitation to prevent finished batch from separating. Separation will occur within a few hours after agitation is stopped.

The mixture was applied to a non-woven substrate (SX285 from Greenbay Nonwovens, 100 grams per square meter) at a weight ratio of about 1:6.5 (substrate: skin-treating composition).

The substrate impregnated with the skin-treating composition was placed in a dryer with wet articles to be dried at a ratio by weight of the impregnated substrate: the dry weight of the articles of 1:150, and the articles dried by hot air while tumbling in contact with the impregnated substrate.

### Example V

An emulsion having the following composition was prepared:

| | % wt. |
|---|---|
| Water | 69.45 |
| PLANTATEX ® LLE | 30.00 |
| KATHON CG-ICP | 0.05 |
| Fragrance* | 0.50 |

*Givaudin UM 046904/00 floral fruity fragrance

A mixing vessel was charged with the water. The PLANTATEX® LLE was added to the water in the mixing vessel with moderate agitation. After a uniform mixture was formed, the KATHON CG-ICP and fragrance were added and incorporated into the emulsion.

The emulsion was supplied to a non-woven substrate (SX 285 product of Greenbay Non-Wovens) at a weight ratio of about 1:7 (substrate:emulsion). The impregnated substrate was introduced into a dryer with fabric articles to be dried at a ratio of weight of impregnated substrate:dry weight of the fabric article of 1:150 and the articles dried in the tumbling dryer at an elevated temperature.

The dried articles were then contacted with human skin and components of PLANTATEX LLE were detected on the human skin. In particular, glycerol oleate, a lipid layer enhancer, was detected on the skin in an amount five times the amount detected on skin which had not been contacted with the treated article.

### EXAMPLE VI

An emulsion having the following composition was prepared:

| | % wt. |
|---|---|
| Water | 65.45 |
| PLANTATEX ® LLE | 30.00 |
| DEHYQUART A-CA | 4.0 |
| KATHON CG-ICP | 0.05 |
| Fragrance* | 0.50 |

*Givaudin UM 046904/00 floral fruity fragrance

A mixing vessel was charged with the water. The PLANTATEX® LLE was added to the water in the mixing vessel followed by DEHYQUART A-CA with moderate agitation. After a uniform mixture was formed, the KATHON CG-ICP and fragrance were added and incorporated into the emulsion.

The emulsion was supplied to a non-woven substrate (SX 285 product of Greenbay Non-Wovens) at a weight ratio of about 1:7 (substrate:emulsion). The impregnated substrate was introduced into a dryer with fabric articles to be dried at a ratio of weight of impregnated substrate:dry weight of the fabric article of 1:150 and the articles dried in the tumbling dryer at an elevated temperature.

The dried articles were then contacted with human skin and components of PLANTATEX LLE were detected on the human skin. In particular, glycerol oleate, a lipid layer enhancer, was detected on the skin in an amount five times the amount detected on skin which had not been contacted with the treated article.

### PLANTATEX LLE (Glycerol Esters Emulsified in Water)

The examples illustrate preparations of emulsions for impregnation on a substrate and tests carried out to determine that the skin-treating components can be transferred through the dried articles to the human skin by contact with the dried articles. The test illustrates that the skin-treatment components can be transferred through the dried articles to the human skin which skin-treatment components are known to benefit human skin.

What is claimed is:

1-18. (canceled)

19. A process for treating human skin comprising:
(a) providing a substrate impregnated with a skin-treating composition comprising:
(i) at least one skin-treating component;
(ii) at least one emulsifier;
(iii) optionally, a carrier for the skin-treating component; and
(iv) optionally, auxiliaries and additives;
(b) providing a clothes dryer which provides tumbling action;
(c) providing at least one article which contacts the skin of a user during use;
(d) inserting said substrate impregnated with said skin-treating composition and said article which contacts the skin of a user during use, into said clothes dryer;
(e) actuating said clothes dryer to produce a predetermined amount of tumbling within the dryer to transfer at least a portion of said skin-treating component to said article to form a skin-treating article;
(f) removing said skin-treating article from said clothes dryer; and
(g) contacting human skin with said skin-treating article whereby said skin-treating component is transferred to the skin.

20. The process of claim 19 wherein said skin-treating component comprises at least one member selected from the group consisting of ethylene glycol distearate, glyceryl oleate, cetyl palmitate, cetrimonium chloride, distearyl ethyl hydroxyethylammonium methosulfate, aluminum chlorhydrate, dicaprylylcarbonate, tocopherol, aloe barbadensis leaf extract, and cyclodextrin.

21. The process of claim 19 wherein said substrate is impregnated with from about 10 grams to about 500 grams of skin-treating composition per square meter.

22. The process of claim 21 wherein said substrate is impregnated with from about 25 grams to about 250 grams of skin-treating composition per square meter.

23. The process of claim 19 wherein said substrate comprises a non-woven material which has been hydro-entangled and/or needle-entangled.

24. The process of claim 19 wherein said skin-treating composition comprises:
(i) from about 5% by weight to about 95% by weight of at least one skin-treating component.
(ii) from about 0.1% by weight to about 40% by weight of at least one emulsifier;
(iii) from 0% by weight to about 95% by weight of a carrier; and
(iv) from 0% by weight to about 35% by weight of auxiliaries and additives;

25. The process of claim 24 wherein said skin-treating composition comprises:
(i) from about 10% by weight to about 75% by weight of at least one skin-treating component.
(ii) from about 0.5% by weight to about 30% by weight of at least one emulsifier;
(iii) from about 20% by weight to about 80% by weight of a carrier; and
(iv) from about 4.5% by weight to about 25% by weight of auxiliaries and additives.

26. The process of claim 25 wherein said skin-treating composition comprises:
(i) from about 15% by weight to about 50% by weight of at least one skin-treating component.
(ii) from about 5% by weight to about 20% by weight of at least one emulsifier;
(iii) from about 35% by weight to about 75% by weight of a carrier; and
(iv) from about 10% by weight to about 20% by weight of auxiliaries and additives.
